(19) United States
(12) Patent Application Publication
(10) Pub. No.: US 2002/0137805 A1
(43) Pub. Date: Sep. 26, 2002

(54) Process for operating a fischer-tropsch reactor

(76) Inventors: Jui-Hsin Ray Huang, Broken Arrow, OK (US); Kym B. Arcuri, Tulsa, OK (US)

Correspondence Address: John E. Vick, Jr., Dority & Manning, P.A., P.O. Box 1449, Greenville, SC, 29602, US

(21) Appl. No.: 09/770,818

(22) Filed: Jan. 26, 2001

(51) Int. Cl. C07C 27/06

(52) U.S. Cl. 518/728; 518/709

(57) ABSTRACT

A process and apparatus for synthesizing hydrocarbons from synthesis gas is disclosed. The process useful for initial start-up of a Fischer-Tropsch reactor system in a slurry bubble column reactor. Synthesis gas comprising hydrogen and carbon dioxide is reacted to form hydrocarbons. A liquid slurry comprising a start-up composition may include polyalpha olefins, astor wax, waxes, and paraffins. The reaction employs a low superficial vapor velocity, a medium to high catalyst concentration, and relatively mild reactor temperatures as compared to conventional Fischer-Tropsch processes.

### FIELD OF THE INVENTION

The field of the invention relates to Fischer-Tropsch (“F-T”) synthesis processes and apparatus for converting synthesis gas (“syngas”) to liquid hydrocarbons. In particular, the invention is directed to improved processes and systems for initial start-up of an F-T reactor.

### BACKGROUND OF THE INVENTION

Slurry hydrocarbon synthesis (HCS) processes are known. In a slurry HCS process a synthesis gas (syngas) comprising a mixture of H₂ and CO is bubbled up as a third phase through a slurry in a reactor in which the slurry liquid comprises hydrocarbon products of the synthesis reaction and the dispersed, suspended solids comprise a suitable Fischer-Tropsch type hydrocarbon synthesis catalyst. Reactors containing such a three phase slurry are sometimes referred to as “bubble columns”, as disclosed in U.S. Pat. No. 5,348,982. Irrespective of whether the slurry reactor is operated as a dispersed or slumped bed, the mixing conditions in the slurry typically will be somewhere between the two theoretical conditions of plug flow and back mixed. The catalyst particles typically are kept dispersed and suspended in the liquid by the lifting action of syngas bubbling up through the slurry.

The slurry liquid hydrocarbon product of the HCS reaction must be separated from the catalyst particles. This typically is accomplished by mechanical filtration in which the slurry is fed into one or more porous filter media that facilitates the liquid passing through, but not the catalyst particles. The hydrocarbon liquid filtrate, which is comprised of longer chain hydrocarbons such as waxes and the like, is then sent to further processing and upgrading.

Bubble columns comprise a liquid medium containing solid particles in suspension, the majority in general being catalytic particles. Such columns comprise at least one means for introducing at least one gas phase of reactants using a distribution mechanism producing bubbles of gas of relatively small diameter These gas bubbles rise in the column, and the reactants are absorbed by the liquid. The gas-absorbed liquid then defuses toward the catalyst. The reactants are converted into gaseous and/or liquid products depending upon the conditions of the reaction and the type of catalyst employed.

The gaseous products comprising the unconverted gaseous reactants and the gaseous products formed during the reaction are collected close to the top of the column. The suspension containing the liquid that forms the suspension of catalyst and liquid products formed during the reaction is recovered via a line located near the upper level of the suspension in the column, as further discussed below in connection with FIG. 1. The solid particles are separated from the liquid using any method which is known to the skilled engineer, for example filtering, to recover the liquid products formed during the reaction.

When using bubble columns as a slurry reactor, however, there are problems that may arise, particularly during the time period in which the reactor is being started or initiated. In reactor start-up, it is important to begin circulating in the reactor a liquid that is capable of dispersing the catalyst. It is usually not practical for a slurry bubble column to simply begin the reaction by pumping syngas into the reactor where there is no wax or long chain hydrocarbons already formed therein.

In the past it has been common to begin reactor start up using a polyalphaolefin (“PAO”) which may be used as a start-up fluid. PAO is a highly paraffinic hydrocarbon. The vapor pressure in the reactor during start-up is also important, and it must be such that the wax will not undesirably convert into a gas at typical synthesis conditions. Furthermore, if the start-up fluid is not chosen carefully, the fluid may deactivate some of the catalyst, which also may cause problems. It is therefore important to begin the reaction sequence with a start-up procedure and composition that comes as close as possible to approximating the actual running conditions produced during the reaction.

In some cases, undesirable foaming may occur in the reactor. When foaming or ineffective degassing occur in the reactor, it reduces the efficiency of reaction. Desirably, a distinct boundary, sometimes called a disengagement zone, should exist in the reactor between the gas near the top of the reactor and the liquid which forms underneath, near the lower portion of the reactor. When foaming occurs, it blurs the line between liquid and gas, undesirably carrying liquid into the upper portions of the reactor.

Studies have indicated that the start up regime of a reactor may determine whether the mode of operation will be “foamy” or a “turbulent bubbling” flow regime. See Bukur et al.: “Flow Regime Transitions in a Bubble Column with a Paraffin Wax as the Liquid Medium”, Ind. Eng. Chem. Res. 1987, 26, pages 1087-1092. It has been found that the start-up procedure employed may determine what type of flow regime ultimately will be obtained by the reactor.

It would be desirable to develop process conditions that facilitate a start-up of the reactor which results in efficient reactor operation. In particular, a start-up procedure or processing condition that avoids the undesirable effects of foaming, ineffective degassing and other reactor disturbances would be very desirable. A composition, system or method of operating a reactor to obtain more efficient and satisfactory results are desired.

### SUMMARY OF THE INVENTION

The invention is directed to an apparatus and process for initiating a hydrocarbon synthesis reaction in a slurry bubble column reactor. The application of the invention may substantially reduce undesirable foaming and ineffective degassing problems that have been observed in such processes. Furthermore, the processes of the invention also may have some positive effects in providing a more desirable mass transfer in the gas to liquid and liquid/catalyst phases of the reaction system.

A synthesis gas is provided to the reactor which comprises both hydrogen and carbon monoxide. A solid particulate hydrocarbon synthesis catalyst is employed by circulating the catalyst to the reactor in a liquid slurry. The liquid slurry further comprises a start-up composition selected from the group of compositions consisting of: polyalpha olefins, astor wax, waxes, and paraffins. The carbon monoxide and hydrogen are reacted to form hydrocarbons in the reactor.

In one application of the invention, the superficial vapor velocity employed is between about 4 and about 12 cm/s. In other applications, the value is about 4-8 cm/s.

In one application of the invention, the catalyst concentration in the liquid slurry is between about 5% and about 20%, and sometimes is within the range of 10-20 vol %.

The overall carbon monoxide conversion in one aspect of the invention is less than about 85% during start-up of the reactor, due in part to reduced temperatures in the reactor. Some embodiments use about 60-85% CO conversion, or less. Temperatures in the reactor may be less than 450 degrees F., and in other applications will be about 380-420 degrees F.

In one particular application of the invention, the viscosity of the liquid slurry in the reactor is less than about 3 centipoise at a temperature of between 380 degrees F. and 420 degrees F. For other applications, the temperature in the reactor is no greater than about 410 degrees F.

### BRIEF DESCRIPTION OF THE DRAWINGS

A full and enabling disclosure of this invention, including the best mode shown to one of ordinary skill in the art, is set forth in this specification. The following Figures illustrate the invention:

FIG. 1 is a schematic of the invention in which a recirculation loop for filtering waxes and returning cleaned catalyst is shown.

Repeat use of reference characters in the present specification and drawings is intended to represent same or analogous features or elements of the present invention.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

The identity of the initial wax medium used in a F-T slurry reaction may have a significant effect on the deactivation rate for the F-T reaction. See Gormley et al.: “Effect of Initial Wax Medium on the Fischer-Tropsch Slurry Reaction”, Applied Catalysis A: General 3846 (1997), pages 1-23.

The Fischer-Tropsch synthesis process is known for converting syngas to liquid hydrocarbons, which can be treated to produce fuel and other specialty products. In commercial practice, the initial start-up of a FT reactor requires a large quantity of start-up fluid, such as polyalphaolefin (PAO), astor wax, and other paraffinic materials. Such start-up fluid is commercially available and usually can be purchased in a large quantity.

Due to the different properties between this type of start-up fluid and the FT wax, the hydrodynamic performance can be varied at the comparable operating conditions. In a 70 barrel per day (“BPD”) F-T reactor there commonly may be some foaming and ineffective degassing problems during the initial start-up. The performance of the F-T reactor is sometimes below expectation, and can be attributed, in part, to poor mass transfer in the gas-liquid and liquid-catalyst phases.

Surprisingly, however, it has been discovered that very beneficial results can be obtained using a start-up FT reactor with a low superficial vapor velocity (4-12 cm/s). Also, better results can be achieved using a high to medium high catalyst concentration (i.e. 5-10%). Furthermore, mild reactor temperatures (less than about 60% CO conversion) also may be desirable.

In general, the superficial vapor velocity may be defined as the gas velocity passing through the reactor column at reaction pressure and temperature. The superficial vapor velocity mentioned herein excludes the column internal content and devices or configuration. The mathematical term of a superficial vapor velocity can be obtained by taking the total gas (vapor) volumetric velocity at temperature and pressure divided by cross-section area of a reactor column.

It has been found that using a low superficial vapor velocity may assist in reducing the foaming tendency in F-T reaction schemes. Furthermore, a medium to high catalyst loading concentration may assist in breaking the foam, and increasing the coalescing effect within the reaction.

Although not required in the practice of the invention, it also has been found that using a relatively mild reactor temperature less than about 400° F. may help to decrease undesirable foaming in the reactor. For example, using a reactor temperature which is low enough so that the degree or amount of conversion of carbon monoxide using the fresh catalyst is no greater than about 60% conversion has been found to be a desirable condition to reduce foaming problems.

The start-up procedures and processing conditions discussed herein may assist in reducing foaming during the initial start-up of a non-F-T wax fluid. This transient stage is developed and designed to ensure that the start-up fluid will be replaced by F-T wax before operating the reactor at the churn turn turbulent flow regime. The churn turbulent flow occurs when the gas velocity is very high so that the bubble momentum becomes very violent in the reactor column. This flow regime includes irregular bubble coalesces and break-up. The shape of bubbles become less spherical during a churn turbulent flow regime due to the turbulent flow mechanism. The churn turbulent flow regime is a function of gas velocity and reactor diameter.

The Fischer-Tropsch synthesis process is known for converting syngas to liquid hydrocarbons, which can be treated to produce fuel and other specialty products. In a commercial practice, the initial start-up of a FT reactor requires a large quantity of start-up fluid, such as polyalphaolefin (PAO), astor wax, or other paraffinic material to suspend the catalyst evenly and react with syngas (H₂ plus CO). Once the reaction takes place at a given temperature, pressure, gas velocity, and catalyst concentration, the start-up fluid can then slowly be replaced with actual F-T products. Consequently, the physical properties of liquid phase in the F-T reactor, such as viscosity, surface tension, and density will be changed accordingly. The longer the reaction takes place, the more the F-T liquid produces in the reactor. The change of these physical properties often is related to the hydronamics of an F-T reactor. This includes gas holdup, flow regime transition, and mass transfer.

Start-up fluids, such as PAO, are commercially available and usually may be purchased in a large quantity for a plant start-up. Due to the different properties between the type of start-up fluids discussed below and the F-T liquid (C₅-C₆₀), the hydrodynamic performance can be varied at the comparable operating conditions.

In some reactors, it has been found that keeping the viscosity of the fluid less than about 3 centipoise at “start of run” conditions (i.e.: “SOR conditions”) is very helpful. The 3 centipoise viscosity is measured at a temperature of about 380° F. to 420° F. In general, the pressure in the reactor during start-up may be between about 300-450 psig, at a temperature of about 400° F. to 420° F., but the invention is not limited in any way to a given pressure level.

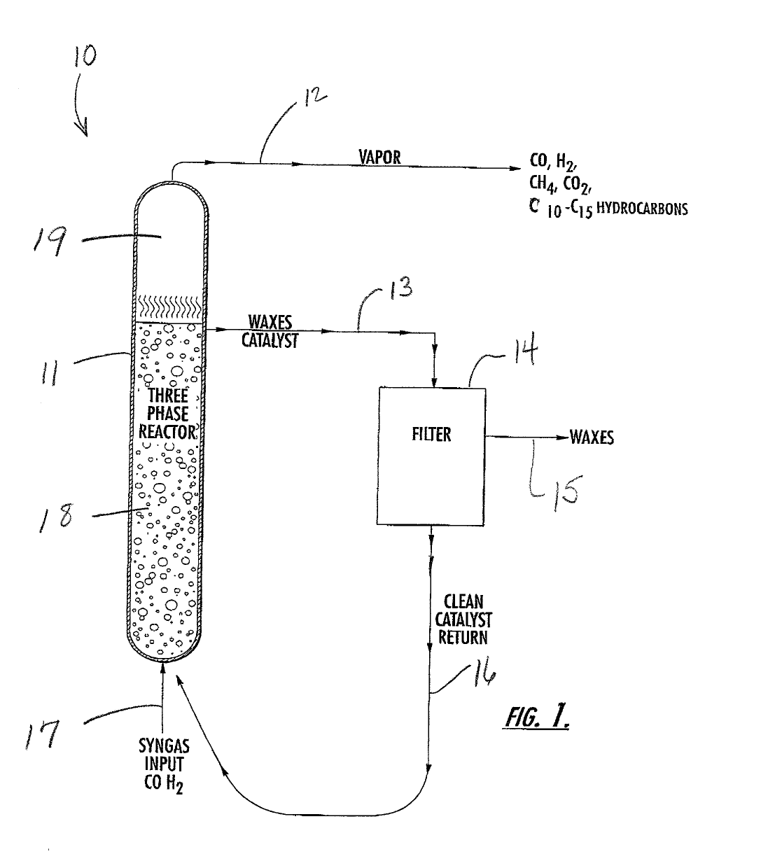

Turning now to FIG. 1, a slurry bubble column reactor (“SBCR”) system 10 is shown. The reactor is a three phase reactor, meaning that it includes reactants in the liquid phase, solid phase and a vapor phase. Synthesis gas (also known as “syngas”) is provided as input to the bottom of the reactor. Syngas comprises a mixture of carbon monoxide and elemental hydrogen. The syngas input 17 is seen near the bottom of the three phase reactor 11. A liquid slurry 18 is provided in the reactor and a gas 19 forms above the liquid phase. A vapor is condensed off from the reactor and an overhead vapor line 12 takes off the more volatile components from the reactor, including carbon monoxide, hydrogen, methane, carbon dioxide, and hydrocarbons having a chain length between about 10 and about 15 carbon atoms (i.e.: C₁₀-C₁₅).

In the reactor system 10, relatively long chained hydrocarbons such as waxes are removed in the liquid phase along heavy hydrocarbon flow line 13. This liquid slurry containing waxes and catalysts is provided into a filter 14 which separates the mixture and provides waxes along the wax removal line 15, and returns clean catalysts along catalyst return line 16. The catalysts, once processed, are returned to the three phase reactor 11 as shown in FIG. 1.

The process of the invention can thus produce essentially paraffinic hydrocarbons in which the fraction with the highest boiling points can be converted to middle distillates (gas oil and kerosine cuts) at high yield using a hydroconversion process such as a catalytic hydroisomerisation and/or hydrocracking.

The gas hourly space velocity (GHSV) is normally in the range 100 to 20,000 volumes of synthesis gas per volume of synthesis gas per volume of catalyst per hour, and preferably less than about 10,000. In one embodiment, an hourly space velocity of 6,000 has proved to be quite efficient.

The catalyst powders employed in this invention usually comprise a Group VIII metal such as iron, cobalt or ruthenium or mixtures thereof on an inorganic oxide support. These catalysts may contain additional promoters comprising Group I, Group II, Group V, or Group VII metals alone or in combination. In some applications, iron based catalysts may be used. Useful catalyst powders of this invention also may comprise cobalt or cobalt and thoria on an inorganic oxide support containing a major amount of titania, silica or alumina. The catalyst may also contain a promoter metal, such as rhenium.

Cobalt-rhenium/titania catalysts exhibit high selectivity in the synthesis of hydrocarbon liquids from carbon monoxide and hydrogen. The catalysts employed in the practice of this invention may be prepared by techniques known in the art for the preparation of such catalysts. The catalyst powder can be prepared by gellation or cogellation techniques. Suitably, however, the metals can be deposited on a previously pilled, pelleted, beaded, extruded, or sieved support material by the impregnation method. In preparing the catalysts, the metals usually are deposited from solution on the support in preselected amounts to provide the desired absolute amounts, and weight ratio of the respective metals, cobalt and rhenium. Suitably, the cobalt and rhenium are composited with the support by contacting the support with a solution of cobalt containing compound or salt, or a rhenium-containing compound, or salt, e.g., a nitrate, carbonate or the like.

The catalyst, after impregnation, is dried by heating at a temperature above 30° C., preferably between 30° C. and 125° C., in the presence of nitrogen, or oxygen, or both, or air, in a gas stream or under partial vacuum.

The catalyst particles, if necessary, are converted to the desired particle size range of nominally 1-200 microns average diameter by crushing, ultrasonic treatment, or other methods known to those skilled in the art. The material can then be sieved, if necessary, to produce a powder that is predominantly within the desired particle size range.

The slurry liquid used in the process is a liquid at the reaction temperature, must be relatively or largely or significantly chemically inert under the reaction conditions and must be a relatively good solvent for CO/hydrogen and possess good slurrying and dispersing properties for the finely divided catalyst.

In a preferred implementation of the process of the invention, the inert liquid phase is advantageously obtained by recycling a portion of a hydrocarbon fraction produced by the reaction; preferably the fraction is the gas oil or kerosine fraction of the hydrocarbons produced by the reaction. In this case, the inert liquid phase which is initially introduced into the reaction zone is supplied from outside (as opposed to a liquid phase produced in the Fischer-Tropsch reaction zone i.e. from the inside) then the liquid phase comprises a portion of a hydrocarbon fraction produced by the reaction which is recycled to said zone. Preferably, the fraction is the gas oil or kerosine fraction.

In the practice of the invention, various types of fluids may be used in the start-up phase of the reaction. As shown in Table 1 below, the physical properties of various start-up fluids are shown. “Callista wax 158” may be obtained from the Shell Oil Company, Lubricants District Sales Office, P.O. Box 4303, Houston, Tex. 77210. “Astor wax 5212” may be purchased from the Astor Corporation, 1100 East Main Street, Titusville, Pa. 16354. “Drakeol 34” is manufactured by Penreco which is a division of the Pennzoil Products Company.

“ISOPAR M” and “V” are aliphatic hydrocarbon fluids which are manufactured by Exxon Chemical Company to meet the low vapor pressure exemption set by the California Air Resources Board for volatile organic compounds in consumer products. “Synfluid PAO 7cSt” is a fully hydrogenated, highly branched isoparaffinic polyalphaolefin. It is manufactured and distributed by Chevron Chemical Company.

TABLE 1
Physical Properties of Various Start Up Fluids

| | | Callista Wax | Astorwax | | | | | | |
|---|---|---|---|---|---|---|---|---|---|
| | Test Method | 158 | 5212 | Drakeol 34 | ISOPAR M | | ISOPAR V | | PAO 7cSt |
| Melting Point, ° F. | ASTM D87 | 160 | 148 | | | | | | |
| Penetration @ 77° F., dmm | ASTM D1321 | 10 | 12 | | | | | | |
| Color, Saybolt | ASTM D156 | 30 | 30 | 30 | 30 | min. | 25 | min. | 30 |
| Oil Content, wt % | ASTM D721 | 0.4 | 0.3 | | | | | | |
| Flash Pensky Martin ° F. | ASTM D93 | 482* | 440 | 480 | 177 | min. | 244 | min. | 514* |
| Viscosity, SUS @ 210° F. | ASTM D2161 | | 44 | 57.9 | | | | | |
| Viscosity, cSt @ 210° F. | ASTM D445 | 5.5 | 5.4 | 9.6 | | | | | 6.94 |
| Viscosity, cSt @ 100° F. | ASTM D445 | | | | 2.4-3.2 | | | | |
| Odor | ASTM D1833 | | 1 | | Pass** | | Pass** | | |
| Pour Point, ° F. | | | | 15 | | | | | |
| Hirschler Molecular Weight | ASTM D2502 | | | 492 | | | | | |
| Carbons as Paraffins | ASTM D3238 | | | 68% | | | | | |
| Carbons as Naphthenes | ASTM D3238 | | | 32% | | | | | |
| Aromatics Content, wt % | ASTM D140.31 | | | | 0.05 | max. | 0.5 | max. | |
| Distillation, IBP ° F. | ASTM D86 | | | | 424 | min. | 505 | min. | |
| Distillation, FBP ° F. | ASTM D86 | | | | 495 | max. | 624 | max. | |
| Sulfur Content, ppm | ASTM D4045 | | | | 5 | max. | | | |

| | | | | | |
|---|---|---|---|---|---|
| Aniline Point, ° F. | ASTM D611 | 185-201 | | | |
| Bromine Index, mg Br/100 g | ASTM D2710 | | 1000 | max. | 109 |
| Total Acid Number | ASTM D974 | | | | 0.01 |

*Cleveland Open Cup — ASTM D92 rather than Pensky Martin - ASTM D93
**BTQAL — 018 Test Method
ASTM D1160 rather than ASTM D86

### EXAMPLE 1

A start-up fluid selected from one of the commercially available fluids provided above is charged into a Continuous Stirred Tank Reactor (CSTR) as illustrated in FIG. 1 using cobalt-based catalyst and conducted at a pressure of 300 psig, a temperature of 410° F., and a Gas Hourly Space Velocity (GHSV) of 6,000. A superficial vapor velocity of less 1 cm/sec. is employed, with a catalyst concentration of less than about 5%. However, the superficial vapor velocity term is not suitable for the CSTR application due to gas inlet location. The example provided herein is provided to screen the start-up fluids rather than for hydrodynamic emphasis.

### EXAMPLE 2

A start-up fluid selected from one of the fluids listed above in Table 1 is used in a 70 barrel per day F-T reactor. The operating conditions are set at favorable range to reduce the foaming tendency. The decrease of gas holdup indicates that the foaming has improved at comparable superficial vapor velocity (10 cm/sec).

A superficial vapor velocity of 10 cm/sec. is employed with a catalyst concentration of less than about 10%. Operating conditions are outlined in Table 1.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

What is claimed is:

1. A process for initiating a hydrocarbon synthesis reaction in a slurry bubble column reactor, comprising:
(a) providing a slurry bubble column reactor;
(b) feeding to the reactor a synthesis gas comprising hydrogen and carbon monoxide;
(c) providing a solid particulate hydrocarbon synthesis catalyst;
(d) circulating said catalyst to the reactor in a liquid slurry, the liquid slurry further comprising a start-up composition selected from the group of compositions consisting of: polyalpha olefins, astor wax, waxes, and paraffins;
(e) reacting the carbon monoxide and hydrogen; and
(f) forming hydrocarbons.

2. The process of claim 1 in which the superficial vapor velocity employed is between about 4 and about 12 cm/s.

3. The process of claim 1 in which the catalyst concentration in the liquid slurry is between about 5% and about 20%.

4. The process of claim 1 in which the overall carbon monoxide conversion is less than about 85% during start-up of the reactor.

5. The process of claim 1 in which the overall carbon monoxide conversion is less than about 60% during start-up of the reactor.

6. The process of claim 1 in which the viscosity of the liquid slurry in the reactor is less than about 3 centipoise, at a temperature of between 380 degrees F. and 420 degrees F.

7. The process of claim 1 in which the capacity of the reactor is about 70 barrels per day.

8. The process of claim 1 in which the temperature in the reactor is no greater than about 410 degrees F.

9. The process of claim 1 in which the Gas Hourly Space Velocity is about 6000.

10. The process of claim 1 in which the start-up composition of the liquid slurry comprises polyalpha olefins, further wherein the composition of the liquid slurry is gradually changed following initial start-up to comprise a smaller percentage of polyalpha olefiins.

11. A process for synthesizing hydrocarbons, comprising:
(a) providing a slurry bubble column reactor;
(b) feeding to the reactor a synthesis gas of hydrogen and carbon dioxide;
(c) providing a liquid slurry, the slurry comprising a start-up composition selected from the group consisting of: polyalpha olefins, astor wax, waxes, and paraffins;
(d) circulating a catalyst in the liquid slurry, the catalyst comprising a concentration of about 5 to about 20 volume percent;
(e) sustaining a superficial vapor velocity of about 2 to about 12 cm/s, and; and
(f) reacting the carbon monoxide and hydrogen to form hydrocarbons.

12. The process of claim 11 in which the reactor temperature is below about 400 degrees F.

13. The process of claim 12 in which the superficial vapor velocity is between about 4 and about 8 cm/s.

14. The process of claim 12 in which the viscosity is less than about 3 centipoise at a temperature of between about 380 to about 420 degrees F.

15. The process of claim 12 in which the catalyst concentration is between about 6 and about 10 volume percent.

16. The process of claim 12 in which the catalyst is a cobalt-based catalyst.

17. The process of claim 12 in which the conversion of carbon monoxide for the overall reaction is less than about 60%.
